SEQUENCE LISTING (2 sequences)

SEQ ID NO: 1 - protein, 321 aa, Gallus gallus
MAKYTRGTVTAFSPFDARADAEALRKAMKGMGTDEETILKILTSRNNAQRQEIASAFKTLFGRDLVDDLKSELTGKFETLMVSLMRPARIFDAHALKHAIKGAGTNEKVLTEILASRTPAEVQNIKQVYMQEYEANLEDKITGETSGHFQRLLVVLLQANRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLYQMIQKDTSGDYRKALLLLCGGDDE

SEQ ID NO: 2 - protein, 313 aa, Gallus gallus
MAAFSPFDARADAEALRKAMKGMGTDEETILKILTSRNNAQRQEIASAFKTLFGRDLVDDLKSELTGKFETLMVSLMRPARIFDAHALKHAIKGAGTNEKVLTEILASRTPAEVQNIKQVYMQEYEANLEDKITGETSGHFQRLLVVLLQANRDPDGRVDEALVEKDAQVLFRAGELKWGTDEETFITILGTRSVSHLRRVFDKYMTISGFQIEETIDRETSGDLEKLLLAVVKCIRSVPAYFAETLYYSMKGAGTDDDTLIRVMVSRSEIDLLDIRHEFRKNFAKSLYQMIQKDTSGDYRKALLLLCGGDDE

(19) United States
(12) Patent Application Publication
(10) Pub. No.: US 2006/0293226 A1
(43) Pub. Date: Dec. 28, 2006

(54) Medicament and use thereof for tumor therapy

(76) Inventor: Wolf Bertling, Erlangen (DE)

Correspondence Address: FISH & RICHARDSON P.C., PO BOX 1022, MINNEAPOLIS, MN, 55440-1022, US

(21) Appl. No.: 10/542,991

(22) PCT Filed: Jan. 19, 2004
(86) PCT No.: PCT/EP04/00362

§ 371 Date: Jul. 24, 2006

(30) Foreign Application Priority Data
Jan. 21, 2003 (DE) 103 02 422.0

(51) Int. Cl. A61K 38/17; A61K 38/19

(52) U.S. Cl. 514/12; 424/85.1

(57) ABSTRACT

The invention relates to a medicament for tumor therapy, containing a first and a second molecule in an effective concentration, the first molecule representing a1) Annexin V or a molecule that is largely similar thereto, or a2) an effective fragment of Annexin V or the molecule that is largely similar thereto, and the second molecule representing b1) a cytokine or a molecule that is largely similar thereto, or b2) an effective fragment of a cytokine or the molecule that is largely similar thereto.

The invention relates to a medicament and use thereof for tumor therapy.

From DE 195 41 284 A1, it is known that Annexin V is suitable for use in the therapy of tumors. This is attributed to the fact that, when Annexin V is administered, the phosphatidylserine-dependent phagocytosis can be influenced.

In accordance with the state of technology, no medicament is known which ensures an effective tumor therapy.

The object of the invention is to remove the disadvantages in accordance with the state of technology. In particular, a medicament and a use thereof for tumor therapy is to be specified.

This object is solved by the features of the claims 1 and 15. Useful embodiments of the inventions result from the features of claims 2 to 14 and 16 to 30.

According to the invention, a medicament for tumor therapy is suggested which contains a first and a second molecule in an effective concentration, wherein the first molecule is

a1) Annexin V or a molecule which is largely similar thereto, or

a2) an effective fragment of Annexin V or the molecule which is largely similar thereto,

and wherein the second molecule is

b1) a cytokine or a molecule which is largely similar thereto or

b2) an effective fragment of a cytokine or the molecule which is largely similar thereto.

Surprisingly, it was shown that a combined administration of Annexin V or similar equal-acting molecules and a cytokine or a similar equal-acting molecule is extremely suitable for the therapy of tumors. Within a short time, for example within just a few days, a reduction of the tumor mass was observed down to the total disappearance of the tumor.

The suggested medicament is also still effective when the first molecule only includes a molecule which is similar to Annexin V or to the cytokine, or an effective fragment of Annexin V or the cytokine, or a molecule which is largely similar to the fragment. A fragment is particularly “effective” when it causes the treated tumor to melt in combination with the respective other molecule. The term “similar molecules” is understood to mean such molecules which to a certain degree have an identity with Annexin V or the cytokine and are effective in combination with the respective other molecule.

The term “cytokine” is understood to mean a protein which is released by a cell and affects the behavior of other cells.

According to an embodiment, an amino acid sequence of the first molecule can correspond to the amino acid sequence of SEQ ID no. 1 or no. 2, or be identical thereto by at least 50%, preferably by at least 60% thereto, particularly preferably by at least 70% thereto, very particularly preferably by at least 80% thereto. The determination of identity can be accomplished for example according to the method of Altschul, S. F. et al. (1997), Nucleic Acids Res. 25:3389-3402.

The term “identity” is understood to mean in this case the extent to which two nucleotides or amino acid sequences are invariant.

With the amino acid sequence of the SEQ ID no. 2, this is an N-terminal deletion mutant of the amino acid sequence of the SEQ ID no. 1, which are missing the eight amino acids 3 to 10, that is the amino acids Lys Tyr Thr Arg Gly Thr Val Thr.

It is useful that the Annexin V is non-human Annexin V, preferably Annexin of the chicken. A comparison of the amino acid sequence of Annexin V of the chicken with human Annexin V shows that both proteins are 78.2% identical. Annexin V of the chicken has a theoretical isoelectrical point (pI) of 5.60 while human Annexin V has a theoretical isoelectrical point of 4.94. The sequence of the human Annexin can be called up under the access number P08756 in the protein data-base “SWISS-PROT.”

The cytokine can be selected from the following group: Interleucine-2, Interleucine-6, Interleucine-7, Interleucine-12, GM-CSF, TNF-α, IL-β.

It has proven to be particularly effective that one administration unit contains 0.05 to 0.5 mg/g_{Tumorweight} on the first molecule. With this, one unit of administration can contain 0.1 to 2.5 mg, preferably 0.5 to 2.0 mg on the first molecule. Furthermore, it preferably contains 50,000 to 1,000,000 International Units, preferably 300,000 to 750,000 International Units on the second molecule. Furthermore, the first and the second molecule are usefully contained in an injection fluid, preferably in a buffered saline solution. The volume of the injection fluid can be 0.5 to 50 ml, preferably 1 to 10 ml.

In addition to the active substance, the medicament can also surround human tumor cells, wherein the tumor cells can be apoptotic and/or necrotic tumor cells. With this, the apoptosis and/or necrosis of the tumor cells can occur spontaneously or can have been induced. Inductors for the apoptosis and/or necrosis may be irradiation of the tumor cells ex-vivo or in-vivo or bringing the tumor cells into contact with cytostatic drugs. Particularly suitable in this connection are chemicals such as H₂O₂ or Staurosporine, medicaments such as adrenocortical steroids, chemotherapeutic substances such as doxorubicin, cis-platinum or hydrox-urea, UVB and UVC radiation, as well as β-, γ- or X-ray radiation. Preferably the apoptotic and/or necrotic tumor cells of the tumor to be treated are brought into contact with the active substance.

In further accordance with the invention, a use of a first molecule, namely

a1) Annexin V or a molecule largely similar thereto, or

a2) an effective fragment of Annexin V or a molecule largely similar thereto,

in combination with a second molecule, namely

b1) Interleucine-2 or a molecule largely similar thereto or

b2) an effective fragment of Interleucine-2 or the molecule largely similar thereto,

is provided for the tumor therapy.

The use for treatment of tumor effusions has been shown to be particularly effective. In particular, the tumor can be a carcinoma of the mamma.

Due to the advantageous embodiments, reference is made to the preceding description. The features mentioned therein can also be considered in the same sense as embodiments of the medicament.

Examples will now be used to describe the invention in more detail based on the drawings. The figures are listed below:

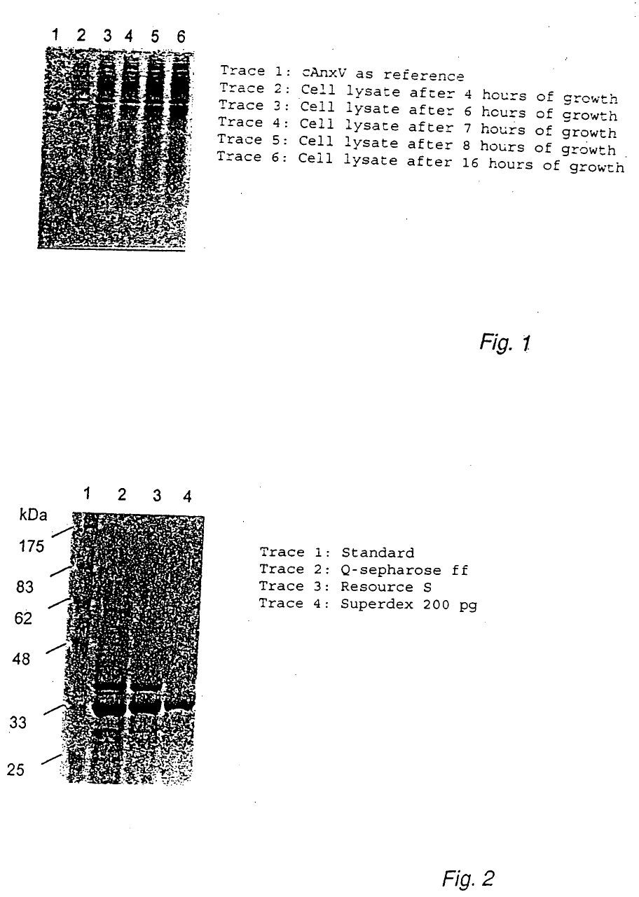

FIG. 1 The expression kinesis of Annexin V of the chicken in transformed Escherichia Coli BL21 (DE3) based on a polyacrylamid gel-electrophoresis and

FIG. 2 A polyacrylamid gel-electrophoresis of samples of the individual purification steps of Annexin V of the chicken from transformed Escherichia Coli BL21 (DE3).

### A. EXPRESSION AND PURIFICATION OF ANNEXIN V OF THE CHICKEN

### 1. Expression of Annexin V of the Chicken

pDJ2-AnxV was used as expression plasmid for the expression of Annexin V of the chicken (cAnxV) which, in addition to the cAnxV-coding gene, also contains an IPTG-inducable lac promoter and a canamycin-resistance cassette. E. coli BL21 (DE3) was transformed with the expression plasmid and expression kineses were performed. With this, 2xYT with 50 mg/l canamycin was used as the culture medium.

To make cAnxV, a 100 ml pre-culture was solubilized with freshly transformed E. coli BL21 (DE3) and shaken at 37° C. for 8 hours. 5 l of the main culture were mixed with 5 ml of the pre-culture and shaken at 37° C. for 16 to 20 hours. A supplement of IPTG was omitted since, in this case, the same expression yields were obtained with and without induction. The cells were then harvested via centrifugation. The cell wet mass was 16 to 21 g. The expression kinesis is shown in FIG. 1. The expression kineses showed that E. coli BL21 (DE3) is suited for an expression of cAnxV with IPTG in the used medium even without induction.

### 2. Purification of Annexin V of the Chicken

The cells obtained as described in number 1 were re-suspended in a buffer A1 (20 mM Tris/HCl pH 7.5, 2 mM EDTA) and broken down with high pressure (Gaulin). Insoluble components of the pulping suspension were removed by high-speed centrifugation. The soluble supernatant containing cAnxV was applied to a Q-sepharose-ff-column equilibrated in buffer A1 (column 1) (25 ml, amersham pharmacia, Freiburg). The elution of the target protein was accomplished via a linear NaCl gradient. The fractions containing cAnxV were pooled and dialyzed against a buffer A2 (50 mM Na-acetate pH 5.6). The dialysate was applied to a resource-S-column (column 2) (6 ml, amersham pharmacia, Freiburg) equilibrated in A2 and cAnxV was eluted with a linear NaCl gradient. The united fractions containing cAnxV were concentrated via ultra-filtration (Pall Filtron, USA) and applied to a Superdex 200 pg-column (column 3) (amersham pharmacia, Freiburg) equilibrated in 10 mM Na-phosphate pH 7.2, 140 mM NaCl. Homogeneous cAnxV was eluted from the column. From 20 g cells (wet mass), 30 mg cAnxV were isolated with a purity exceeding 95%. FIG. 2 uses a polyacrylamid gel-electrophoresis to show the results of the purification using columns 1 to 3.

As an alternative, instead of the specified columns, Q-sepharose XL (amersham pharmacia, Freiburg) can be used for column 1, SP-sepharose HP (amersham pharmacia, Freiburg) can be used for column 2 and/or sephacryl S200 HR (amersham pharmacia, Freiburg) can be used for column 3.

As an alternative, instead of the size elimination chromatography (SEC) performed via column 3, a hydrophobic column can be used. In this case, the fractions containing cAnxV which elute from column 2 are united and solid ammonium sulphate is added, up to 1.5 M. The protein solution is applied to a phenylsepharose ff-column (15 ml, amersham pharmacia, Freiburg) and cAnxV is eluted with a linear gradient of 1.5 to 0 M ammonium sulphate.

### B. TUMOR THERAPY

During an individual treatment attempt, a patient's melanoma with a size of approximately 2 cm was injected with Annexin V in a 1 ml buffered saline solution as per sequence protocol SEG ID NO: 1, together with 500,000 International Units of Interleucine-2. Already after just a few days, a distinct melting away of the melanoma was observed.

According to a further therapy method, it is also possible to first extract tumor cells from the patient. The extracted tumor cells are mechanically dissociated; the number of cells is determined. Then the cells are irradiated with 100 Gray so that the tumor cells are transformed into apoptotic or necrotic tumor cells. 10×10⁶ of the apoptotic or necrotic tumor cells are then mixed with a buffered saline solution which contains 1 mg Annexin V as per sequence protocol SEQ.1 or SEQ.2. An incubation then follows. Immediately prior to the injection, 500,000 International Units of Interleucine-2 are added. The mixture containing apoptotic tumor cells, Annexin V and Interleucine-2 is then injected into the patient intradermal or intracutaneous. Also this caused the treated tumor to already melt away significantly after just a few days.

To increase the efficiency of the previously stated therapy method and to discourage a recurrence, the injection can be repeated for example on day 21, 42 and on later days or during the second, third and sixth week.

As proof of the particular effectiveness of a combined administration of cytokine and Annexin V, the supernatants of peritoneal macrophages as well as dendritic cells extracted from bone narrow are each incubated in a medium for 24 hours. The respective amounts (in pg/ml) of released cytokines, namely TNF-α, IL-1β, are then determined via ELISA. The experiments are repeated under identical conditions, wherein the medium is incubated together with irradiated tumor cells (ITC), with Annexin V (AxV) and with irradiated tumor cells and Annexin V. With the co-incubation, the ratio of ITC:phagocytes was 5.:1. The obtained results were evaluated as per Students t Test, wherein *=p<0.01; **=p<0.005.

The following table compares the obtained results.

| | Macrophages | | | | Dendritic Cells | | | |
|---|---|---|---|---|---|---|---|---|
| Cytokine | Medium | | Medium + AxV | | Medium | | Medium + AxV | |
| (pg/ml) | / | ITC | / | ITC | / | ITC | / | ITC |
| TNF-α | 125 ± 15 | 335 ± 30 | 117 ± 24 | 978 ± 48** | 267 ± 137 | 392 ± 79 | 245 ± 121 | 426 ± 78 |
| IL-1β | 21 ± 5 | 44 ± 7 | 14 ± 1 | 322 ± 85* | 10 ± 4 | 46 ± 4 | 8 ± 3 | 47 ± 3 |

*P < 0.05
**P < 0.01

The results clearly show that, after co-incubation with irradiated tumor cells, the secretion of post-inflammatory cytokine, namely TNF-α, IL-1β, is clearly increased by macrophages. This effect is drastically increased with a co-incubation of macrophages with tumor cells which have been irradiated and treated with Annexin V.

1-30. (canceled)

31. A medicament for tumor therapy, comprising an effective concentration of a first and a second molecule,
wherein the first molecule is Annexin V, a fragment thereof having functional Annexin V activity, or a polypeptide having at least 80% sequence identity to Annexin V and having functional Annexin V activity,
wherein the second molecule is a cytokine, a fragment thereof having functional cytokine activity, or a polypeptide having at least 80% sequence identity to a cytokine and having functional cytokine activity.

32. The medicament of claim 31, wherein the Annexin V has at least 85% sequence identity with SEQ ID NO: 1 or 2.

33. The medicament of claim 31, wherein the Annexin V has at least 90% sequence identity with SEQ ID NO: 1 or 2.

34. The medicament of claim 31, wherein the Annexin V has at least 95% sequence identity with SEQ ID NO: 1 or 2.

35. The medicament of claim 31, wherein the Annexin V has the sequence shown in SEQ ID NO: 1 or 2.

36. The medicament of claim 31, wherein the Annexin V is a non-human Annexin V.

37. The medicament of claim 36, wherein the non-human Annexin V is a chicken Annexin V.

38. The medicament of claim 31, wherein the cytokine is selected from the group consisting of Interleucine-2, Interleucine-6, Interleucine-7, Interleucine-12, GM-CSF, TNF-α, and IL-1β.

39. The medicament of claim 31, wherein one unit of administration comprises 0.05 to 0.5 mg/g_{Tumorweight} of the first molecule.

40. The medicament of claim 31, wherein one unit of administration comprises 0.1 to 2.5 mg of the first molecule.

41. The medicament of claim 40, wherein one unit of administration comprises 0.5 to 2.0 mg of the first molecule.

42. The medicament of claim 31, wherein one unit of administration comprises 50,000 to 1,000,000 International Units of the second molecule.

43. The medicament of claim 42, wherein one unit of administration comprises 300,000 to 750,000 International Units of the second molecule.

44. The medicament of claim 31, wherein the medicament is formulated as an injectable fluid.

45. The medicament of claim 44, wherein the medicament is formulated in a buffered saline solution.

46. The medicament of claim 31, wherein one unit of administration comprises 0.5 to 50 ml.

47. The medicament of claim 46, wherein one unit of administration comprises 1 to 10 ml.

48. The medicament of claim 31, further comprising apoptotic and/or necrotic tumor cells.

49. The medicament of claim 48, wherein the tumor cells are human tumor cells.

50. The medicament of claim 48, wherein the tumor cells are in contact with the first and second molecule.

51. A method of reducing the mass of a tumor, comprising:
contacting the tumor with the medicament of claim 31.

52. The method of claim 51, further comprising the step of monitoring the tumor for a reduction in mass.

53. The method of claim 51, wherein the tumor is a tumor effusion.

54. The method of claim 51, wherein the tumor is a mamma carcinoma.

55. The method of claim 51, wherein the medicament is injected into the tumor.

56. The method of claim 55, wherein the volume of the medicament injected is 0.5 to 50 ml.

57. The method of claim 56, wherein the volume of the medicament injected is 1 to 10 ml.

58. The method of claim 51, wherein the medicament further comprises apoptotic and/or necrotic tumor cells.

59. The method of claim 51, wherein the tumor cells are human tumor cells.

60. The method of claim 51, wherein the tumor cells are apoptotic and/or necrotic tumor cells from the contacted tumor.
